(19) United States
(12) Patent Application Publication
(10) Pub. No.: US 2001/0007656 A1
(43) Pub. Date: Jul. 12, 2001

(54) TREATMENT OF DIABETES WITH TRANSCRIPTION FACTOR GENE

(76) Inventors: FATIMA BOSCH, CERDANYOLA (ES); ALFONS VALERA, CERDANYOLA (ES)

Correspondence Address: Y ROCKY TSAO, FISH & RICHARDSON, 225 FRANKLIN STREET, BOSTON, MA, 021102804

(21) Appl. No.: 08/748,469

(22) Filed: Nov. 8, 1996

(51) Int. Cl. A61K 31/70; A01N 43/04; A61K 48/00

(52) U.S. Cl. 424/93.2; 514/44; 424/450; 536/23.1

(57) ABSTRACT

The invention features a process of treating a diabetic patient, which comprises the step of administering to said patient a DNA segment which includes a transcription factor gene and a promoter sequence, said promoter sequence being operably linked to said transcription factor and being effective for the expression of a therapeutically effective amount of the transcription factor in said patient.

### BACKGROUND OF THE INVENTION

The activity of functional genes in the mammalian genome is tightly regulated by a very broad class of proteins called “transcription factors”. Transcription factors are proteins that have DNA binding domains capable of binding to specific DNA sequence elements or recognition sites. The binding of transcription factors to such DNA elements (i.e., motifs) in the promoter region of a gene results in the turning on of transcriptional activity, leading to the generation of a messenger RNA and, subsequently, the production of the protein encoded by the gene in the cell. This process is collectively described as gene expression.

Transcription factors may be active on their own or act in concert with other proteins, transcriptional modulators, or co-factors to activate transcription. Transcriptional modulators can also act to inhibit the activity of transcription factors to down-regulate gene expression.

Thus, whether a target gene is expressed, and to what extent it is expressed, is regulated at two levels: (1) by the amount of a specific transcription factor produced; and (2) by the activity of the transcription factor expressed. These two levels are in turn controlled at the level of cellular signalling, wherein the level of transcription factors produced is influenced by the metabolic or proliferative demands placed on the cell by the hormonal milieu (e.g., in response to insulin, growth factors). See e.g., Calkhoven, C. F., et al., Biochem. J. 317:329-342 (1996).

In the physiological regulation of carbohydrate metabolism in mammals, glucose is known to modulate the transcription of many genes, especially those involved in hepatic metabolism (Vaulont, S., et al., FASEB J. 8:28-35 (1994)). The promoters of these genes associated with glucose homeostasis and utilization contains a class of DNA elements by which the expression of the genes are responsive to changes in glucose concentration, e.g., glucose-responsive elements (GlcRE). See, e.g., Vaulont, S., et al., FASEB J. 8:28-35 (1994) and Girard, J., et al., FASEB J. 3:36-42 (1994). Within the GlcRE is an imperfect motif for an “E Box” motif, with the consensus sequence CACGTG. This motif is the recognition sequence for a family of transcription factors, such as TFEB (Carr, C. S., et al., Mol. Cell Biol. 10:4384-4388 (1990); and Cuif, et al., J. Biol. Chem. 268:13769 (1993)), TFEC (Zhao, G., et al., Mol. Cell Biol. 13:4505-4512 (1992)), USF 2/FIP (Shih, et al., J. Biol, Chem. 267:13222 (1992); and Sirrito, M., et al., Gene Exp. 2:231-240 (1992)), CBF1/CPI (Cai, M., et al., Cell 61:437-446 (1990)) PHO4 (Ogawa, N., et al., Mol. Cell Biol. 10:2224-2236 (1990)), MLTF/USF (Gregor, P. D., et al., Genes & Dev. 4:1730-1740 (1990); and Luscher, et al., Genes & Dev. 4:2025 (1990)), c-Myc (Stanten, L., Nature 310:423 (1984); and Blackwell, T. K., et al., Science 250:1149-1151 (1990)), Max (Blackwood, E. M., et al., Science 251:1211-1217 (1991)), Mad (Ayer, D. E., et al., Cell 72:211-222 (1993)), Mxi (Zervos, A. S., et al., Cell 72:223-232 (1993)), and TFE3 (Beckmann, H., et al., Genes & Dev. 4:167-179 (1990); and Liu, et al., J. Biol. Chem. 268:12787 (1995)). This GlcRE is also closely related functionally to the carbohydrate response elements, ChoRE in the S14, L-type pyruvate kinase (Bergot, M. O., et al., Nucleic Acid Res. 20:1871-1878 (1992) and Shih, H., et al., J. Biol. Chem. 267:13222-13228 (1992)) and fatty acid synthase genes.

The c-Myc gene is expressed at high levels in the liver throughout development (Stanlon, B., et al., Genes & Dev. 6:2235-2247 (1992)) and, to a lesser extent, in the adult liver (Xu, L., et al., Mol. Cell Biol. 11:6007-6015 (1991)). In addition, insulin regulates the transcription of c-Myc in rat hepatoma cells (Messina, J. L., J. Biol. Chem. 266:17985-18001 (1991)). It is thus possible that the transcription factor c-Myc may have a role in the regulation of expression of genes involved in hepatic carbohydrate metabolism (Valera, A., et al., FASEB J. 9:1067-1078 (1995)).

The present invention describes the utility of targeting a transcription factor transgene for the prevention and treatment of diabetic and insulinopenic states.

### SUMMARY OF THE INVENTION

An aspect of the invention features a process of treating a diabetic patient. The process includes the step of administering to the patient a DNA segment containing a transcription factor gene and a promoter sequence, in which the promoter sequence is operably linked to the transcription factor gene and is effective for the expression of a therapeutically effective amount of transcription factor in the diabetic patient. It was unexpected that the expression of transcription factor in a diabetic patient would have helped normalize the patient's glucose level in the absence of or independent of the insulin level. The term “therapeutically effective amount” means an amount of the expressed transcription factor which is sufficient to effect glucose uptake into cells, tissue, or organ of the patient, and can be determined without undue experimentation.

As used herein, the term “transcription factor” refers to a protein with a DNA binding domain capable of recognizing and binding to DNA sequences containing a central consensus sequence CACGTG. An example of such a transcription factor is c-Myc. The transcription factor coding sequence of the DNA segment can be the same or substantially the same as the coding sequence of the endogenous transcription factor coding sequence as long as it encodes a functional transcription factor proteins. Indeed, the DNA segment can also be the same or substantially the same as the transcription factor gene of a non-human species as long as it encodes a functional transcription factor protein. The transcription of the transcription factor gene in the DNA segment is preferably under the control of a promoter sequence different from the promoter sequence controlling the transcription of the endogenous coding sequence, e.g., a promoter sequence which remains activated or induced during diabetic conditions in the patient, such as elevated levels of glucose, glucagon, triglyceride, or free fatty acids. Examples of such promoter sequences include the phosphoenolpyruvate carboxykinase (“PEPCK”) promoter and the myosin light chain (“MLC”) promoters, e.g., MLC1, MLC2, and MLC1/3 promoters.

In one embodiment, the DNA segment is introduced to the diabetic patient in cells, wherein the cells are treated in vitro to incorporate therein the DNA segment and, as a result, the cells express in vivo in the diabetic patient a therapeutically effective amount of transcription factor. The DNA segment can be introduced into the cells by a viral vector, e.g., a retroviral vector.

In another embodiment, the DNA segment is directly introduced to the diabetic patient, e.g., not contained within a cell. The DNA segment can be introduced in a vector. Examples of suitable vectors include viral vectors (e.g., retroviral vectors, adenoviral vectors, adeno-associated viral vectors, sindbis viral vectors, and herpes viral vectors), plasmids, cosmids, and yeast artificial chromosomes. The DNA segment can also be introduced as infectious particles, e.g., DNA-ligand conjugates, calcium phosphate precipitates, and liposomes.

Also contemplated within the scope of this invention are the DNA segments described herein, as well as vectors and cells containing such a DNA segment.

Other features and advantages of the present invention will be apparent from the brief description of the drawings, the detailed description of the invention, and from the claims.

### BRIEF DESCRIPTION OF THE DRAWING

The figures are first briefly described:

FIG. 1 is a diagrammatic representation of the construction of a DNA vector carrying a transcription factor gene for expression into liver cells.

FIG. 2 is a diagrammatic representation of the construction of a DNA vector carrying a transcription factor gene for expression into muscle cells.

### DETAILED DESCRIPTION OF THE INVENTION

The methods of making and using DNA segments to practice the therapeutic process of this invention are well within the ability of a person or ordinary skill in the art. Unless defined otherwise, all technical and scientific terms used herein have the same meaning as commonly understood by one of ordinary skill in the art to which this invention belongs. Also, all publications cited herein are incorporated by reference.

The therapeutic process of the invention allows for the overexpression (e.g., at a higher level than pretreatment) of transcription factor in a diabetic patient. The overexpression of transcription factor in the diabetic patient results in the uptake of glucose into the cells (e.g., liver cells) within the patient.

What is meant by “DNA segment” herein is any exogenous DNA construct which includes a sequence encoding for a functional transcription factor, and the transcription factor is expressed by the cells into which the DNA segment is introduced. The DNA segment can be introduced into both the somatic and germ cells or only into some of the somatic cells of the patient, or cells expressing the DNA segment can be introduced ex vivo into the patient. The DNA segment, therefore, may or may not be an integral part of the patient's chromosome, and if the DNA segment is integrated into a chromosome, it may or may not be located at the same site as its corresponding endogenous gene sequence.

The DNA segment used to practice the therapeutic process includes a transcription factor gene or its complementary DNA (“cDNA”), whose expression is driven by a promoter which is expressed during diabetic conditions. Examples of suitable promoters include the general constitutively active promoters/enhancers, e.g., the β-actin promoter (Kawamoto, et al., Mol. Cell Biol. 8:267-272 (1988); Morishita, et al., Biochem. Biophys. Acta 1090:216-222 (1991)), cytomegalo virus (“CMV”) and SV40 promoters, (Okayama, et al., Mol. Cell Biol. 3:280-289 (1983); and Boshart, et al., Cell 41:521-530 (1985)), or retroviral long terminal repeat sequences (LTR) (Crystal, R. G., Science 270:404-410 (1995)), and strong tissue specific constitutive promoters, e.g., the muscle-specific myosin light chain promoters (Lee, et al., J. Biol. Chem. 267:15875-15885 (1992); and Greishammer, et al., Cell 69:79-93 (1992)) and the liver specific albumin promoter (Heckel, et al., Cell 62:447-456 (1991)).

The promoter is comprised of a cis-acting DNA sequence which is capable of directing the transcription of a gene in the appropriate environment, tissue, context, and in response to physiological regulators, e.g., hormones, glucose, and intermediary biochemical metabolizers. The expression of a transgene (e.g., a transcription factor gene or its CDNA sequence) can be regulated. Examples of inducible promoters include the glucagon inducible phosphoenolpyruvate carboxykinase promoter (Valera, et al., Proc. Natl. Acad. Sci. USA, 91:9151-9154 (1994)), the divalent metal inducible metallothionein promoter (Karin, et al., Proc. Natl. Acad. Sci. USA 80:4040-4044 (1993)), and the tetracycline inducible promoter (Grossen, et al., Science 268 (1995)).

The genomic DNA sequence and cDNA sequences of the transcription factor gene for various species have been reported. Examples include the transcription factor DNA sequence for the major late transcription factor/upstream stimulating factor (MLTF/USF), Myc, Max, Mad, Mxi, the immunoglobulin enhancer binding proteins TFE3, TFEB, TFEC, the Fos interacting protein USF2/FIP, CBFI/CPI, and PHO4.

Examples of cells targeted for overexpression of transcription factor include hepatocytes from the liver (Peng, et al., Proc. Natl. Acad. Sci. USA 85:8146 (1988); Wolff, et al., Proc. Natl. Acad. Sci. USA 84:3344 (1987); and Wilson, et al., Proc. Natl. Acad. Sci. USA 85:3014 (1988)), myoblast and myocytes from the muscle (Salminer, et al., Human Gene Therapy 2:15-26 (1991)), stem cells of the bone marrow (Bakx, et al., Human Gene Therapy 2:301-306 (1991); Williams, et al., Nature 310:476-480 (1984); Lim, et al., Proc. Natl. Acad. Sci. USA 86:8892-8896 (1989); Demarguoy, et al., Human Gene Therapy 3:3-10 (1992); and Wieder, Human Gene Therapy 2:323-326 (1991)), fibroblasts from the skin (Wolff, et al., Proc. Natl. Acad. Sci. USA 87:2877-2881 (1990); Morgan, et al., Science 237:1476-1479 (1987); and Green, Scientific American 265:96-102 (1991)), neuronal cells from the brain (Price, et al., Proc. Natl. Acad. Sci. USA 84:156-160 (1987); and Renfranz, et al., Cell 66:713-729 (1991)), and endothelial cells (Nabel, et al., Science 244:1342-1344 (1989)).

To practice the therapeutic process of this invention, one can use vectors (both viral and non-viral DNAs, e.g., plasmids, cosmids, and yeast artificial chromosomes) and other gene delivery systems available for either the in vitro expression into cells utilized in ex vivo implantation or direct in vivo delivery of a transcription factor gene into the cells or tissues of a patient. Detailed guidance is provided below:

Viral Vectors for the Delivery of a Transcription Factor Gene

Viral vectors can be used for the delivery of a transcription factor gene. Examples of viral vectors include recombinant retroviral vectors, recombinant adenoviral vectors, recombinant adeno-associated viral vectors, sindbis viral vectors, and recombinant herpes viral vectors.

(a) Recombinant Retrovirus Vectors

The genome of recombinant retroviral vector is comprised of long terminal repeat (“LTR”) sequences at both ends which serve as a viral promoter/enhancer and a transcription initiation site and a Psi site which serves as a virion packaging signal and a selectable marker gene (e.g., a neomycin resistance gene). An example of such vector is pZIP-NeoSV (Cepko, et al., Cell 53:103-1062 (1984)). The transcription factor gene can be cloned into a suitable cloning site in the retroviral genome. Expression is under the transcriptional control of the retroviral LTR. The vector will drive the constitutive expression of transcription factor in the appropriate cell type. The level of expression is dictated by both the promoter strength of the LTR. Tissue selectivity is determined by both the origin of the viral genome (e.g., sarcoma virus, leukemia virus, or mammary tumor virus) and the cell line used to package the virus.

Specific modifications in the LTR sequence to improve the level of expression of the cloned gene have been described (Hilberg, et al., Proc. Natl. Acad. Sci. USA 84:5232-5236 (1987); Holland, et al., Proc. Natl. Acad. Sci. USA 84:8662-8666 (1987); and Valerio, et al., Gene 84:419-427 (1989)). The transcription factor gene can also be cloned into the vector linked to an internal promoter as an expression cassette (Crystal, R. G., Science 270:404-410 (1995)). The use of an internal promoter has also been shown to confer an additional level of control on gene expression (Lai, et al., Proc. Natl. Acad. Sci. USA 86:10006-10010 (1989); and Scharfmann, et al., Proc. Natl. Acad. Sci. USA 88:4626-4630 (1991)). Examples of internal promoter are strong constitutive promoters, e.g., the β-Actin promoter (Kawamoto, et al., Mol. Cell Biol. 8:267-272 (1988); Morishita, et al., Biochem. Biophys. Acta 1090:216-222 (1991); and Lai, et al., Proc. Natl. Acad. Sci. USA 86:10006-10010 (1989)), the H-2k promoter (Schuh, et al., Nature 345:757-760 (1990); and Jat, et al., Proc. Natl. Acad. Sci. USA 88:5096-5100 (1991)), and the CMV and SV40 promoters (Miller, et al., Biotechniques 7:980-990 (1989)). The promoter can also be an inducible-regulatable promoter, e.g., the mouse metallothionein promoter (Karin, et al., Proc. Natl. Acad. Sci. USA 80:4040-4044 (1983)), the tetracycline inducible promoter (Gossen, et al., Science 1766-1769 (1995); and Efrat, et al., Proc. Natl. Acad. Sci. USA 92:3576-3580 (1995)), a tissue specific promoter/enhancer, e.g., the liver specific mouse albumin promoter (Heckel, et al., Cell 62:447-456 (1991)) and PEPCK promoter (Valera, et al., FASEB J. 8:440-447 (1994)), the muscle specific myosin light chain promoters (Lee, et al., J. Biol. Chem. 267:15875-15885 (1992); Shen, et al., Mol. Cell Biol. 11:1676-1685 (1991); Lee, et al., Mol. Cell Biol. 14:1220-1229 (1994); Grieshammer, et al., Cell 69:79-93 (1992); and Donoghue, et al., Genes & Dev. 2:1779-1790 (1988)), the alpha-myosin heavy chain promoter (Molkentin, et al., J. Biol. Chem. 268:2602-2609 (1993)), or the fat specific adipocyte P2 promoter (Graves, et al., Genes & Dev. 5:428-437 (1991); Ross, et al., Proc. Natl. Acad. Sci. USA 87:9590-9594 (1990)). Examples of such retroviral vectors incorporating an internal promoter include vLPGKSN (Valera, et al., Eur. J. Biochem. 222:533-539 (1994)) and mLBSN (Ferrari, et al., Human Gene Therapy 6:733-742 (1995)).

Recombinant retroviruses capable of transducing the transcription factor gene are produced by transfecting the recombinant retroviral genome(s) into a suitable (helper-virus free) amphotropic packaging cell line. Examples of virus packaging cell lines include PA317 and Psi CRIP (Cornetta, et al., Human Gene Therapy 2:5-14 (1991); Miller, et al., Mol. Cell Biol. 6:2895-2902 (1986); and Cone, et al., Proc. Natl. Acad. Sci. USA 81: 6349-6353 (1984)). The transfected virus packaging cell line will package and produce recombinant retroviruses, shedding them into the tissue culture media. The retroviruses are harvested and recovered from the culture media by centrifugation (Compere, et al., Mol. Cell Biol. 9:6-14 (1989)). The viruses are resuspended in a suitable buffer, e.g., 10 mM HEPES, and stored at −70°C. or in liquid nitrogen.

Retrovirus vectors can offer a wide host range and tissue tropism with the appropriate choice of internal promoter and virus packaging cell line. Selective targeting is achieved by modification of the envelope protein produced by the packaging cell line. For example, through the generation of a chimeric envelope protein with a single chain variable fragment from a monoclonal antibody recognizing the human low density lipoprotein receptor, it was possible to efficiently target infection of the cells expressing the receptor (Somia, et al., Proc. Natl. Acad. Sci. USA 92:7570-7574 (1995)).

(b) Recombinant Adenovirus Vectors

Adenovirus can be used as a vector for transducing a transcription factor expression cassette. A number of adenovirus vectors have been developed for the transduction of genes into cells (Berkner, et al., BioTechniques 6:616-629(1988)). Constitutive high level expression of the transduced gene products has been achieved. These vectors have the inherent advantage over the retroviral vectors in not requiring replicating cells for infection, making them suitable vectors for somatic gene therapy (Mulligan, R. C., Science 260:926-932 (1993)).

Replication defective adenoviruses lacking the E1 region of the genome have been developed which will accommodate an insertion of 7.5 kilobases of foreign DNA (Crystal, R. G., Science 270:404-410 (1995); Logan, et al., Proc. Natl. Acad. Sci. USA 81:3655-3659 (1994); Freidman, et al., Mol. Cell Biol. 6:3791-3797 (1986); Levrero, et al, Gene 101:195-202 (1991); and Imler, et al., Human Gene Therapy 6:71-721 (1995)). These replication defective recombinant adenoviruses can be propagated by transfecting the genome into cells engineered to express the E1 genes (Jones, et al., Cell 16:683 (1979); and Berkner, et al., BioTechniques, 6:616-629 (1988)). This system allows the production of adenovirus particles at high titer (e.g., up to 10¹³/ml), which greatly enhances infection efficiency by enabling a higher multiplicity of infection (Crystal, R. G., Science 270:404-410 (1995)).

Strategies for generating adenoviral recombinants have been described (Berkner, et al., BioTechniques 6:616-629 (1988)). An example is the use of the plasmid pMLP6 (Logan, et al., Proc. Natl. Acad. Sci. USA 81:3655-3659 (1984)) which carries the Adenovirus 5 genome with the E1 region deleted. Digestion with the restriction endonucleases BglII and RsaI will produce a linearized plasmid that retains only the left most 194 bp of the Adenovirus 5 genome. An expression cassette containing a regulated tissue specific promoter region, e.g., the PEPCK promoter (Valera, et al., FASEB 8:440-447 (1994)), linked to a DNA fragment encoding transcription factor with compatible 3′ and 5′ ends (modified by appropriate linker ligations and then subjected to appropriate restriction endonuclease digestion as described in Maniatis, et al., Molecular Cloning—A Laboratory Manual (Cold Spring Harbor Laboratory, Cold Spring Harbor, 1989) can be cloned into the Adenovirus 5 plasmid. The entire recombinant Adenovirus genome is then generated by mixing the linearized Adenovirus 5-PEPCK-Transcription factor plasmid with a subgenomic fragment of Adenovirus DNA representing the 3.85-100 map units (prepared by digesting the In340 viral genome with ClaI or XbaI) (N. E. Biolabs, Beverly, Mass.) (Berkner, et al., BioTechniques 6:616-629 (1988)). The DNAs are then transfected into human kidney 293 cells (Graham, et al., J. Gen. Virol. 36:59-72 (1977)) as described in Berkner, et al., Nuc. Acid. Res. 11:6003-6020 (1983). Intermolecular recombination across appropriate segments of the plasmid and the subgenomic fragment of adenoviral DNA will result in the production of replication defective recombinant adenoviral genomes carrying the PEPCK-Transcription factor chimeric gene. The recombinant genomes will emerge from the 293 cell lines as packaged viral particles shedded into the medium. Modifications of this design have resulted in high level expression vectors (Berkner, et al., BioTechniques 6:616-629 (1988)) by incorporating regions of the major late promoter and the tripartite leader elements (Berkner, et al., Nuc. Acid Res. 11:6003-6020 (1983); and Logan, et al., Proc. Natl. Acad. Sci. USA 81:3655-3659 (1984)) in the vector construction.

The use of recombinant adenoviruses for delivery of genes into cells of the airway in humans and animals have been described (Reviewed in Crystal, R. G., Science 270:404-410 (1995); and Katkin, et al, Human Gene Therapy 6:985-995 (1995)). The feasibility for transducing genes associated with glycogen metabolism, using adenovirus-mediated transfer in primary rat hepatocytes and myoblast in culture, has also been described (Baque, et al., Biochem. J. 304 (Pt 3):1009-1014 (1994); and Gomez-Foix, et al., J. Biol. Chem. 267:25129-25134 (1992)).

(c) Recombinant Adeno-Associated Viruses

Adeno-associated virus (“AAV”) can also be used as a vector for transducing a transcription factor expression cassette. AAV offers the advantage that it has not been implicated in the etiology of any disease, and its site specific integration on human chromosome 19 has been shown not to interfere with host gene expression or promote gene rearrangements (Kotin, et al., Proc. Natl. Acad. Sci. USA 87:2211-2215 (1990); and Samulski, et al., Eur. Mol. Biol. Org. J. 10:3941-3950 (1991)). As with adenoviruses, AAV is capable of infecting post-mitotic cells, thereby, making it a suitable vector for delivery of genes to somatic cells.

The AAV genome contains two genes, rep and cap, and inverted terminal repeats (ITR) sequences (Hermonat, et al., J. Virol. 51:329-339 (1984)). Recombinant AAV vectors are constructed by replacing the rep gene, the cap gene, or both with a transcription factor gene expression cassette (Hermonat, et al., Proc. Natl. Acad. Sci. USA 81:6466-6470 (1984)). The sole sequence needed for AAV vector integration is the terminal 145 base ITR (Muzyczka, Curr. Top. Microbiol. Immunol. 158(97):97-129 (1992)). Such vectors are available in the plasmid form (Tratschin, et al., Mol. Cell Biol. 5:3251-3260 (1985); Lebkowski, et al., Mol. Cell Biol. 8:3988-3996 (1988); and McLaughlin, et al., J. Virol. 62:1963-1973 (1988)).

The recombinant AAV genomes can be packaged into AAV particles by co-transfection of the vector plasmid and a second packaging plasmid carrying the rep and cap genes into an adenovirus-infected cell. Such particles have been shown to efficiently transduce heterologous genes into a number of mammalian cell lines (Tratschin, et al., Mol. Cell Biol. 5:3251-3260 (1985); Lebkowski, et al., Mol. Cell Biol. 8:3988-3996 (1988); McLaughlin, et al., J. Virol. 62:1963-1973 (1988); and Flotte, et al., Am. J. Respir. Cell. Mol. Biol. 7:349-356 (1992)).

In addition to using an expression cassette, high levels of expression of genes linked directly to the endogenous AAV p40 promoter has been demonstrated (Wondisford, et al., Mol. Endocrinol. 2:32-39 (1988)).

(d) Herpes Virus Vectors

Herpes virus (“HSV”) vectors constitute a unique system for the delivery of genes into cells of neuronal lineage (Anderson, et al., Cell Mol. Neurobiol. 13:503-515 (1993). HSV-derived vectors infect post-mitotic neurons and produce an established latent infection in some cell types making it a suitable system for somatic gene therapy (Leib, et al., BioEssays 15:547-554 (1993)).

Strategies for the generation of HSV vectors and recombinant viruses suitable for the transduction of the transcription factor gene have been described (Leib, et al., BioEssays 15:547-554 (1993)). The general method extensively used for mutagenizing endogenous viral genes (Post, et al., Cell 25:227-232 (1981)) can be applied for the introduction of exogenous transcription factor genes into the HSV genome.

The transcription factor expression cassette is cloned into a plasmid containing a portion of the HSV genome such that at least 300 bp flank the 5′- and 3′ ends of the cassette (Breakfield, et al., New Biol. 3:203-218 (1991); and Efstathiou, et al., Brit. Med. Bull. 51:45-55 (1995)). The plasmid is transfected into permissive cells in culture along with the full length HSV DNA (Geller., et al., Proc. Natl. Acad. Sci. USA 87:8950-8954 (1990)). Homologous recombination and DNA replication will result in the generation of recombinant HSV genomes that are packaged into novel virus particles by the cell. Through several round of plaque purification, a recombinant virus carrying the transcription factor expression cassette can be identified for large scale production.

Defective HSV vectors have been successfully used to transfer exogenous genes into neurons in vitro and in vivo (Geller, et al., Proc. Natl. Acad. Sci. USA 87:1149-1153 (1990); Geller, et al., Science 241:1667-1669 (1988); and Efstathiou, et al., Brit. Med. Bull. 51:45-55 (1995)). A variety of constitutive promoters have been used including the lytic cycle HSV promoters, the Rous sarcoma virus LTR, the human cytomegalo virus (HCMV) IE promoters, and the neurofilament and PGK promoters for transient expression. Long term expression have been obtained using the Moloney murine leukemia virus LTR, HSV LAT promoter, HCMV IE promoter fused to the LAT promoter elements, and the neuron specific enolase promoter. These vectors have been reported to be useful for transduction of genes into cells of non-neuronal origin, e.g., mouse hepatocytes (Efstathiou, et al., Brit. Med. Bull. 51:45-55 (1995); and Miyanohara, et al., New Biol. 4:238-246 (1992)).

e) Sindbis Virus Vectors Expressing Transcription Factor

Sindbis virus-based vectors are intended as self-amplifying systems to enhance expression of exogenous genes in mammalian cells (Herweijer, et al., Human Gene Therapy 6:1161-1167 (1995)). In this system, the subgenomic sequence coding for the structural proteins are replaced by the expression cassette of the transgene, e.g., transcription factor (Huang, et al., Virus Genes 3:85-91 (1989); and Bredenbeek, et al., J. Virol. 67:6439-6446 (1993)). The recombinant sindbis virus is generated by placing the entire genome under the control of the bacteriophage T7 or SP6 promoters to enable transcription of the (+) strand RNA in vitro (Herweijer, et al., Human Gene Therapy 6:1161-1167 (1995)) The resultant RNA genomes are then used to transfect target cells (Xiong, et al., Science 243:1188-1191 (1989)). Infectious viruses are produced by infecting with a helper virus (Bredenbeek, et al., J. Virol. 67:6439-6446 (1993)). Modifications of this design using the Rous sarcoma virus LTR to direct the transcription of the non-structural genes have been described (Herweijer, et al., Human Gene Therapy 6:1161-1167 (1995)).

To generate a recombinant Sindbis virus vector, the luciferase gene cloned into the unique XbaI site in the vector pSin-Lux (Herweijer, et al., Human Gene Therapy 6:1161-1167 (1995)) is replaced by the transcription factor cDNA or an expression cassette encoding transcription factor upon appropriate restriction endonuclease modifications. See Sambrook, et al., Molecular Cloning—A Laboratory Manual (Cold Spring Harbor Laboratory, 1989).

Sindbis virus vectors have been successfully used to transduce foreign genes into 3T3 cells (mouse fibroblast), 293 cells (human kidney cell line), HepG2 cells (human hepatoma cell line), and primary rat myoblasts (Herweijer, et al., Human Gene Therapy 6:1161-1167 (1995)).

In Vivo Delivery of a Transcription Factor Gene by Viral Infection

Viral vectors can be used to deliver the transcription factor coding sequence into the cells, tissues, and organ of diabetic patients by in vivo infection. In in vivo infection, the recombinant viral vector is administered to the organism in order to result in a general systemic infection or organ/tissue specific infection of the patient. For example, intravenous injection of recombinant retrovirus or aerosol administration of recombinant adenoviral vectors results in infection of the epithelial lining of the respiratory tract (Rosenfield, et al., Science 252:431-434 (1991); and Hsu, et al., J. Infectious Dis. 166:769-775 (1992)); stereotaxic inoculation of recombinant herpes simplex viruses have resulted in infection of select regions of the brain (Fink, et al., Human Gene Therapy 3:11-19 (1992)); and infection of mitotically active (regenerating) liver by direct injection or administration through the portal vein of recombinant retroviruses has resulted in persistent expression of the infecting genome (Kalenko, et al., Human Gene Therapy 2:27-32 (1991); and Rettinger, et al., Proc. Natl. Acad. Sci. USA 91:1460-1464 (1994)).

Ex Vivo Delivery of a Transcription Factor Gene

(a) In Vitro Transduction of Cells in Culture

Primary cells and cell lines are grown in the recommended media supplemented with the appropriate growth factors, serum, and antibiotics. The cells are transduced with the transcription factor gene either by direct infection with a recombinant viral vector described above or by non-viral delivery means, e.g., DNA-mediated transfection. Recombinant DNA expression cassettes comprising of cellular promoters/enhancers and regulatory regions operably linked to the transcription factor genes/cDNAs designed for expression in target mammalian tissues in the form of plasmids, linearized DNA fragments, or viral DNA/RNA vectors are prepared and purified as described in Sambrook, et al., Molecular Cloning—A Laboratory Manual (Cold Spring Harbor Laboratory, 1989). DNA can be introduced into cells by DNA-mediated transduction following one of the following methods: calcium phosphate precipitation, DEAE-Dextran method, electroporation (Ausudel, et al., Current Protocols in Molecular Biology (Wiley-Interscience, 1987)), or ofectin or protoplast fusion (Sandra-Goldin, et al., Mol. Cell Biol. 1:743-752 (1981)). Where the selectable marker is on a separate plasmid, the calcium phosphate co-precipitation method (Ausudel, et al., Current Protocols in Molecular Biology (Wiley-Interscience, 1987)) is used.

Twenty-four hours after DNA-mediated transfection, the cells in culture are trypsinized and replated in selection media at a density of 1/10. Clonal cell line that have inherited the selectable marker are picked by ring cloning, expanded in culture, and analyzed for the inheritance of the transfected gene of interest by PCR (Innis, et al., PCR Protocols: A Guide to Methods and Applications (Academic Press, 1990)) and Southern blot analysis (Southern, J. Mol. Biol. 98:503 (1975)) of genomic DNA prepared from the clonal cells/cell lines. Expression of the transfected transcription factor gene are examined by Northern blot analysis (Sambrook, et al., Molecular Cloning—A Laboratory Manual, (Cold Spring Harbor Laboratory, 1989) of total RNA by immunoblot analysis with transcription factor-specific antisera and by transcription factor activity assay (Valera, et al., Eur. J. Biochem. 222:533-539 (1994); Davidson, et al, Arch. Biochem. Biophys. 253:156-167 (1987)).

(b) Ex Vivo Modification of Cells for Implantation

The technologies for virus and DNA-mediated gene transduction into mammalian cells (e.g., primary cells and cell lines) allow for ex vivo cellular engineering. These engineered cells can serve as metabolic factories (Newgard, Diabetes 43:341-350 (1994); Hughes, et al., Proc. Natl. Acad. Sci. USA 89:688-692 (1992); and Newgard, C. B., J. Lab. Clin. Med. 122:356-363 (1993)), as novel drug delivery systems (Kasid, et al., Proc. Natl. Acad. Sci. USA 87:473-477 (1990); and Chen, et al., Human Gene Therapy 6:917-926 (1995)), as surrogate tissues or organs (Mendell, J. R., et al., N. Engl. J. Med. 333:832-838 (1995); and Rhim, et al., Science 263:1149-1152 (1994)), or as neo-organs (Thompson, et al., Proc. Natl. Acad. Sci. USA 86:7928-7932 (1989)); and Culliton, Science 246:747-749 (1989)) upon appropriate implantation into the patient.

For example, hepatocytes can be isolated from the liver (Ponder, et al., Proc. Natl. Acad. Sci. USA 1217-1221 (1991); and Pages, et al., Human Gene Therapy 6:21-30 (1995)), committed to short term culture (Pages, et al., Human Gene Therapy 6:21-30 (1995)), and then transduced with a viral or plasmid vector carrying the expression cassette comprising of the transcription factor cDNA under the transcriptional control of a liver specific promoter. The genetically modified hepatocytes are then harvested and transplanted into a patient either by infusion of the cells into the portal vein (Wilson, et al., Proc. Natl. Acad. Sci. USA 87:6437-8441 (1990)) or by intrasplenical introduction (Ponder, et al., Proc. Natl. Acad. Sci. USA 88:1217-1221 (1991)). In mice, genetically modified hepatocytes introduced intrasplenically were shown to replace up to 80% of the diseased liver (Rhim, et al., Science 263:1149-1152 (1994)). In dogs, a 5% replacement of the liver mass with hepatocytes transduced with the human α1-antitrypsin expressing retrovirus resulted in the expression of the human peptide for up to 30 days (Kay, et al., Proc. Natl. Acad. Sci. USA 89:89-93 (1992)). Similarly, hypercholesterolemia in Watanabe heritable hyperlipidemic rabbits were transiently corrected by implantation of hepatocytes transduced with a retrovirus capable of directing the expression of a functional low density lipoprotein (“LDL”) receptor (Wilson, Proc. Natl. Acad. Sci. USA 87:8437-8441 (1990)). An internal liver specific promoter could enhance sustained level of expression of the transgene.

In another example, myoblasts can be isolated from muscle biopsies (Mendell, et al., N. Engl. J. Med. 832-838 (1995)), expanded in culture, and genetically modified to express high, levels of transcription factor by transfection with DNA comprising of the transcription factor gene under the transcription control of a strong muscle specific promoter/enhancer or infected with a muscle specific recombinant retrovirus (Ferrari, et al., Human Gene Therapy 6:733-742 (1995)). The transcription factor expressing myoblasts can then be transferred into muscle by direct injection of the cells. Previous experience with murine myoblast have demonstrated that the injected myoblasts will fuse into pre-existing multinucleate myofibrils (Dhawan, et al., Science 254:1509-1512 (1991); and Barr, et al., Science 254:1507-1509 (1991)). The differentiated muscle fibers will maintain a high level of expression of the transgene (Yao, et al., Proc. Natl. Acad. Sci. USA 89:3357-3361 (1992)).

In still another example, human fibroblasts can be modified by receptor mediated or retroviral mediated gene transfer (Veelken, et al., Human Gene Therapy 5:1203-1210 (1994); and Chen, et al., Human Gene Therapy 6:917-926 (1995)) to overexpress transcription factor. The cells are then embedded into collagen coated lattices of expanded polytetrafluoroethylene (Gore-Tex) fibers as previously described (Thompson, et al., Proc. Natl. Acad. Sci. USA 86:7928-7932 (1989); and Moullier, et al., Nature Genetics 4:154-159 (1993)). Adsorption of heparin-binding growth factors-1 to the collagen lattices, upon implantation into the peritoneal cavity, will induce vascularization and formation of a neo-organ. Such neo-organs were reported to produce a sustained expression of transgenes in mice (Salvetti, et al., Human Gene Therapy 6:1153-1159 (1995)) and dogs (Moullier, et al., Nature Med. 1:353-357 (1995)). Such neo-organs comprising of fibroblast or other types of cells overexpressing transcription factor can serve to normalize blood sugars in the insulinopenic state.

In Vivo Non-Viral Delivery of a Transcription Factor Gene Into Patients

The non-viral transcription factor gene constructs can also be targeted in vivo to specific tissue or organs, e.g., the liver or muscle, in patients. Examples of such delivery systems include receptor mediated endocytosis, liposome encapsulation, or direct insertion of non-viral expression vectors.

(a) Receptor Mediated Endocytosis

By receptor mediated endocytosis, the transcription factor gene can be delivered to specific cells, e.g., hepatocytes in the liver (Wu, et al., J. Biol. Chem. 266:14338-14342 (1991); and Wilson, et al., J. Biol. Chem. 267:963-967 (1992)). Using asialoorosomucoid coupled to poly-L-lysine, soluble transcription factor DNA complexed to this molecule can be selectively delivered to hepatocytes in the liver via binding to the asialoglycoprotein receptor followed by endocytosis. The ligand-DNA complex is administered intravenously. A transient alleviation of hypercholesterolemia has been obtained with this technology by delivering functional LDL receptors into LDL receptor deficient rabbits (Wilson, et al., J. Biol. Chem. 267:963-967 (1992)). Other DNA-ligand conjugates have been developed, e.g., transferrin-polylysine-DNA complexes (Wagner, et al., Proc. Natl. Acad. Sci. USA 87:3410-3414 (1990); Wagner, et al., Proc. Natl. Acad. Sci. USA 89:7934-7938 (1992); and Wagner, et al., Proc. Natl. Acad. Sci. USA 89:6099-6103 (1992)), surfactant B-polylysine-DNA complexes (Baatz, et al., Proc. Natl. Acad. Sci. USA 91:2547-2551 (1994)), and anti-thrombomodulin-polylysine-DNA complexes (Trubetskoy, et al., Bioconjug. Chem. 3:323-327 (1992)).

(b) Liposome Encapsulation

Successful in vivo gene transfer has been achieved with the injection of DNA, e.g., as a linear construct or a circular plasmid, encapsulated in liposomes (Ledley, Human Gene Therapy 6:1129-1144 (1995) and Farhood, et al., Ann. N.Y. Acad. Sci. 716:23-35 (1994)). A number of cationic liposomes amphiphiles are now in development (Ledley, Human Gene Therapy 6:1129-1144 (1995); Farhood, et al., Ann. N.Y. Acad. Sci., 716:23-35 (1994).

Intratracheal administration of cationic lipid-DNA complexes was shown to effect gene transfer and expression in the epithelial cells lining the bronchus (Brigham, et al., Am. J. Respir. Cell Mol. Biol. 8:209-213 (1993); and Canonico, et al., Am. J. Respir. Cell Mol. Biol. 10:24-29 (1994)). Expression in pulmonary tissues and the endothelium was reported after intravenous injection of the complexes (Brigham, et al., Am. J. Respir. Cell Mol. Biol. 8:209-213 (1993); Zhu, et al., Science, 261:209-211 (1993); Stewart, et al., Human Gene Therapy 3:267-275 (1992); Nabel, et al., Human Gene Therapy 3:649-656 (1992); and Canonico, et al., J. Appl. Physiol. 77:415-419 (1994)). The expression cassettes for transcription factor in linear, plasmid or viral DNA forms can be condensed through ionic interactions with the cationic lipid to form a particulate complex for in vivo delivery (Stewart, et al., Human Gene Therapy 3:267-275 (1992)).

Other liposome formulations, for example, proteoliposomes which contain viral envelope receptors proteins, i.e., virosomes, have been found to effectively deliver genes into hepatocytes and kidney cells after direct injection (Nicolau, et al., Proc. Natl. Acad. Sci. USA 80:1068-1072 (1993); Kaneda, et al., Science 243:375-378 (1989); Mannino, et al., Biotechniques 6:682 (1988); and Tomita, et al., Biochem. Biophys. Res. Comm. 186:129-134 (1992)).

(c) Direct Injection

Direct injection of transcription factor DNA expression vectors, e.g., into the muscle or liver, either as a solution or as a calcium phosphate precipitate (Wolff, et al., Science 247:1465-1468 (1990); Ascadi, et al., The New Biologist 3:71-81 (1991); and Benvenisty, et al., Proc. Natl. Acad. Sci. USA 83:9551-9555 (1986)), provides alternative technology for delivering the transcription factor expression cassettes into tissues of recipients.

Microinjection of the transcription factor DNA segment, e.g., as a linear construct or a circular plasmid, into the pronucleus of a zygote or two-cell embryos that could transmit the transgene to subsequent generations constitutes an approach to achieving germ line gene therapy (Hogan, et al., Manipulating the Mouse Embryo, Cold Spring Harbor Laboratory, 1986); Brinster, et al., Proc. Natl. Acad. Sci. USA 82:4438-4442 (1985)). Another method for achieving germ-line gene therapy is effecting gene transfection and homologous recombination in embryonic stem cells (Thomas, et al., Cell 51:503-512 (1987); and Capecchi, Science 244:1288-1292 (1989)).

The following are examples which demonstrates certain aspects of how to practice the therapeutic process of this invention. It is believed that one skilled in the art, based upon the description herein, can utilize the invention to its fullest extent. The specific examples set forth below are, therefore to be construed as merely illustrative and not limitative of the remainder of the disclosure whatsoever.

### EXAMPLE 1

Generation of PEPCK/c-Myc Construct

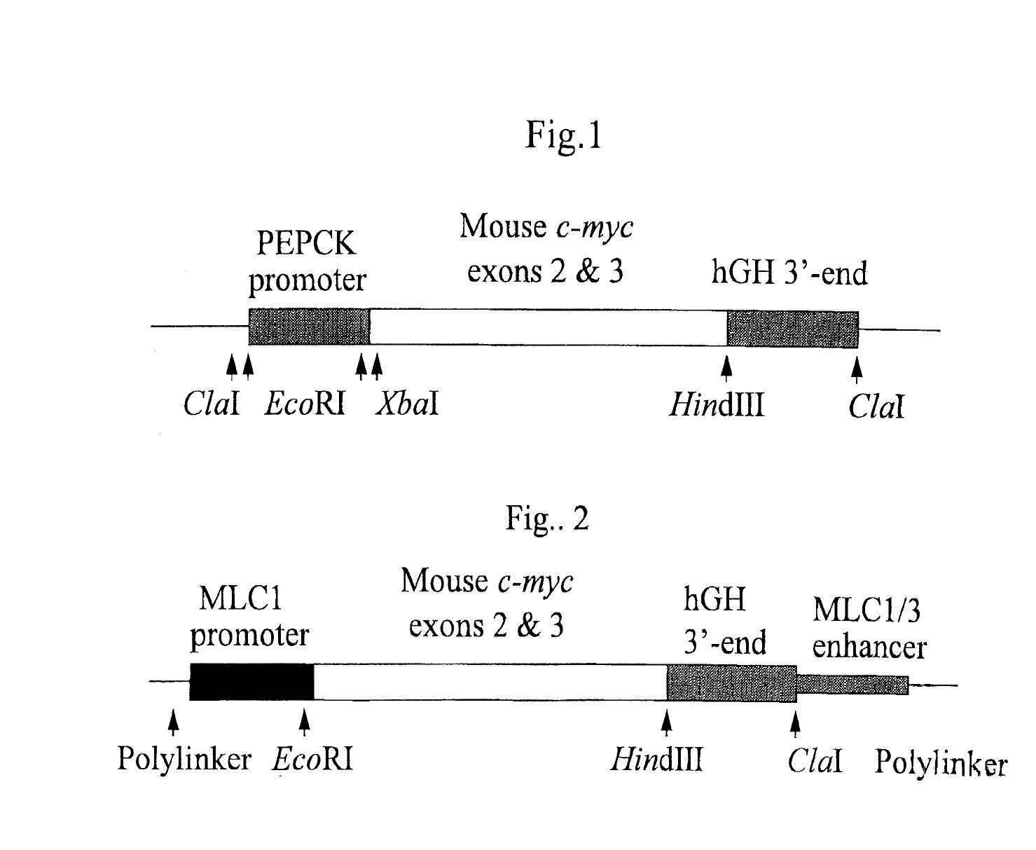

To obtain the PEPCK/c-Myc chimeric gene, the 2.8 kb Xbal-HindIII fragment of the mouse c-Myc gene, which contains the coding exons 2 and 3 of the gene, was used. Stanton, et al., Nature 310:423 (1984). This fragment was introduced at the Xbal-HindIII sites of the plasmid p12N, a pBR322-based plasmid that contains a ClaI polylinker. The −485 to +73 bp Xbal-BgIII fragment of the rat PEPCK promoter was introduced at the Xbal-BamHI sites of the BluescriptR plasmid (Stratagene). Yoo-Warren, H., Proc. Natl. Acad. Sci. USA 80:656 (1983). Afterward, the EcoRI-EcoRI fragment containing 450 bp of PEPCK promoter was introduced at the EcoRI site of the p12N in front of the c-Myc gene. Finally the HindIII-XhaI 3-end fragment of the human growth hormone gene (hGH) was introduced at the end of the PEPCK/c-Myc chimeric gene, previously digested with HindIII (at the end of the c-Myc gene) and SalI (in the ClaI polylinker). See FIG. 1.

### EXAMPLE 2

Production of PEPCK/c-Myc Transgenic Mice

The general procedures for microinjection of the chimeric gene were as previously described. See Hogan, B., Manipulating the Mouse Embryo:A Laboratory Manual (Cold Spring Harbor Laboratory Press, Cold Spring Harbor, N.Y. b 1986). A 4.1 kb ClaI-ClaI fragment, containing the entire chimeric gene, was microinjected into fertilized eggs. Fertilized mouse eggs were flushed from the oviducts of superovulated C57BL6/SJL mice 6-8 h after ovulation. Male pronuclei of the fertilized eggs were injected with 2 pl of DNA solution (approximately 2 ng/ul), and viable embryos were reimplanted in the oviducts of pseudopregnant mice. At 3 weeks of age, the animals were tested for the presence of the transgene by Southern blot of DNA tail samples. Mice were fed ad libitum with a standard diet (Panlab, Barcelona, Spain) and maintained under a light-dark cycle of 12 h (lights on at 8:00 a.m.). When stated, mice were starved for 24 hours. Animals were killed, and samples were taken between 9 and 10 a.m. In the experiments described below, male mice two to four months old were used.

### EXAMPLE 3

Production of Diabetes in Transgenic Mice, and Analysis of Transgene Expression

Diabetes was induced by injection through the jugular vein of doses of 2 mg of streptozotocin (Stz; Sigma Chemicals, St. Louis, Mo.) per 10 g of body weight on two consecutive days. Stz was dissolved in a 10 mM sodium citrate solution with 0.9% NaCl (pH 4.5) immediately before administration. Mice were used 7 days after Stz treatment. Diabetes was assessed by measuring glycemic, glucosuric, and ketonuric levels (Accutrend and Gluketur Test; Boehringer Mannheim, Germany), as well as insulin blood levels. The mice were killed, and samples were taken between 9 and 10 a.m. In the experiments described below male mice, (F₂ generations) of age 4-8 weeks, were used. Valera, et al., FASEB J. 9:1067 (1995)

A 1.7 Kb transcript from the expression of the c-Myc transgene was detected by Northern blot analysis of total RNA (Sambrook, et al., Molecular Cloning—A Laboratory Manual, Cold Spring Harbor Laboratory, 1989) obtained from the liver of transgenic mice treated with Stz.

The effects of overexpression of c-Myc after Stz treatment in the liver of transgenic mice were assessed as follows:

a) Expression of Glucokinase

While no glucokinase mRNA transcripts were detected by Northern blot analysis in Stz-treated control mice, Stz-treated transgenic mice expressed high levels of glucokinase in mRNA. Thus, in the absence of insulin, the increase in c-Myc transcription factor appeared to mimic the effects of the hormone on the expression of glucokinase, acting either directly or through the activation of other transcription factor(s). The increase in the expression of glucokinase was parallel to the activation of the enzyme. Glucokinase activity was determined in liver samples as described in Valera, A., et al., FASEB J. 9:1067-1078 (1995). The enzyme activity in diabetic control mice was extremely low. However, glucokinase activity of Stz-treated transgenic mice was even higher than that noted in healthy control mice (See Table I).

TABLE I

| | | Stz-Treated | Stz-Treated |
|---|---|---|---|
| Parameter | Control | Control | Transgenic |
| Glucokinase, mU/mg of | 24.6 ± 3.3 | 4.8 ± 2.9 | 39.5 ± 3.6 |
| protein | | | |
| Glucose 6-P, nmol/g of liver | 211 ± 15 | 68 ± 9 | 218 ± 17 |
| Glycogen, mg/g of liver | 41.4 ± 5.6 | 5.1 ± 2.7 | 37.9 ± 6.4 |
| Pyruvate kinase U/mg of | 0.21 ± 0.03 | 0.12 ± 0.03 | 0.35 ± 0.06 |
| protein | | | |
| Lactate, μmol/g of liver | 0.16 ± 0.03 | 0.04 ± 0.02 | 0.23 ± 0.4 |

b) Intracellular Concentration of Glucose-6-Phosphate

The reduction of glucokinase activity led to a decrease (about 70%) in the intracellular concentration of glucose 6-phosphate in diabetic control mice compared with healthy controls. In contrast, Stz-treated transgenic mice presented high levels of this metabolite, similar to those of healthy control mice (see Table I). The concentration of hepatic glucose-6-PO4 was measured as previously described in Valera, A., et al., FASEB J. 9:1067-1078 (1995).

c) Glycogen Storage in the Liver

Glucose 6-phosphate is a substrate for the synthesis of glycogen as well as an allosteric activator of glycogen synthase. During diabetes, because of the lack of insulin and the increase in glucagon, glycogen synthase is phosphorylated and inactive (Larner, J., Adv. Enzymol. Relat. Areas Mol. Biol. 63:173 (1990); and Roach, P. J., FASEB J. 4:2961 (1990)). As expected, no glycogen was stored in the liver of diabetic control mice. However, Stz-treated transgenic mice showed levels of glycogen similar to those in control healthy mice (Table I), probably resulting, at least in part, from the increase of glucose 6-phosphate. Thus, the expression of the PEPCK/c-Myc chimeric gene prevented the loss of glucokinase activity and the glucose storage during diabetes in the transgenic mice. Hepatic glycogen levels were measured using the α-amyloglucosidase method (Keppler, D., et al., Glycogen in Methods of Enzymatic Analysis, Bergmeyer, H. U. ed, vol. 6, pp 11-18, Verlag Chemie GmBH, Weinheim, Germany, 1981)

d) Glucose Utilization

Glucose utilization was also increased in the transgenic mice in both fed and starved conditions (Valera, et al., FASEB J. 9:1067 (1995)). The L-type pyruvate kinase (L-PK) is a main regulatory enzyme of glycolysis (Pilkis, et al., Annu. Rev. Physiol. 54:885 (1992)). When L-PK gene expression was analyzed, a 3-fold increase in the 3.2-kb mRNA transcript (Cuif, et al., Mol. Cell. Biol. 12:4852 (1992)) was observed in Stz-treated transgenic mice compared with control mice. The increase in L-PK mRNA was concomitant with an induction of L-PK activity, which was even higher than that noted in fed control mice. c-Myc prevented the development of diabetic alterations after Stz treatment, at least in part through its ability to induce hepatic glucose uptake and utilization. Pyruvate kinase activity was determined as described in Valera, A., et al., FASEB J. 9:1067-1078 (1995).

e) Gluconeogenesis and Ketogenesis

The effects of Stz treatment on hepatic gluconeogenesis and ketogenesis in these animals were also analyzed. Glucose production from gluconeogenic precursors by hepatocytes in primary culture from Stz-treated transgenic mice was lower than that of Stz-treated control mice and similar to that of healthy control mice. An induction of ketogenesis is a common feature of untreated insulin-dependent diabetes mellitus (McGarry, et al., Annu. Rev. Biochem. 49:395 (1980); and McGarry, et al. Science 258:766 (1992)). The concentration of ketone-bodies in the incubation medium of Stz-treated transgenic mice was similar to that of healthy control mice. These results indicated that ketogenesis was blocked (see Table II) in Stz-treated mice expressing the PEPCK/c-Myc chimeric gene. The β-hydroxybutyrate (ketoacid) levels of serum and in the incubation medium of hepatocytes were measured by the β-hydroxybutyrate dehydrogenase technique (Boehringer Mannheim, Germany).

f) Glycemia

As expected, diabetic control mice had high levels of blood glucose (about 4-fold increase over healthy controls), whereas Stz-treated transgenic mice showed a dramatic reduction of hyperglycemia (Table II). All these mice had very low levels of circulating insulin (Table II). In addition, the concentration of serum β-hydroxybutyrate was normalized in diabetic transgenic mice compared with diabetic controls (Table II). Transgenic mice also showed normal levels of circulating triacylglycerols and free fatty acids, which were markedly increased during diabetes (Table 2). Furthermore, diabetic control mice lost body weight, and a 25% reduction was noted 12 days after Stz treatment (from 23.3±0.6 g to 17.4±0.8 g; n=19). The normalization of serum parameters in the Stz-treated transgenic mice was also accompanied by the maintenance of body weight (from 20.8±0.6 g to 21.7±0.9 g; n=22). These results indicated that transgenic mice developed resistance to the establishment of the common alterations noted in diabetic animals.

TABLE II

| | | Stz-Treated | Stz-Treated |
|---|---|---|---|
| Parameter | Control | Control | Transgenic |
| Glucose, mg/dl | 189 ± 8 | >600 | 243 ± 12 |
| Insulin, ng/ml | 1.92 ± 0.25 | <0.2 | <0.2 |
| β-Hydroxybutyrate mmol/liter | 0.39 ± 0.08 | 3.22 ± 0.34 | 0.56 ± 0.11 |
| Triacylglycerols, mg/dl | 134 ± 10 | 352 ± 31 | 138 ± 18 |
| Free fatty acids, mmol/liter | 0.72 ± 0.11 | 2.08 ± 0.35 | 0.69 ± 0.13 |

In summary, all these results indicated that c-Myc overexpression in the liver of transgenic mice prevented the development of diabetic alterations after Stz treatment not only by inducing hepatic glucose uptake and utilization, but also by blocking the diabetic activation of gluconeogenesis and ketogenesis.

### EXAMPLE 4

Generation of the Rat Myosin Light Chain-1/Rat c-Myc (MLC1/c-Myc) Construct

To obtain the MLC1/c-Myc chimeric gene, a 3.7 kb EcoRI-ClaI fragment containing the entire coding sequence of the mouse c-Myc gene (exon 2 & 3) and the hGH polyadenylation signal from the plasmid PEPCK/c-Myc (FIG. 1) is used. A 1.5 kb EcoRI/HindII fragment of the MLC1 promoter, including the cap site and spanning 105 bp of untranslated sequences, is fused to the 5′ end of the c-Myc cassette. Then, a 0.9 kb SphI/HindIII genomic fragment of the MLC1/3 gene, containing a strong muscle-specific enhancer (Donoghue, et al., Genes & Dev. 2:1779-1790 (1988) is introduced in the transgene at the 3′ end of the hGH fragment by standard recombinant DNA manipulation to ensure high levels of expression in skeletal muscle. The resulting plasmid is designated pMLC1/c-Myc. See FIG. 2.

### Other Embodiments

It is to be understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A process of treating a diabetic patient, which comprises the step of administering to said patient a DNA segment which includes a transcription factor gene and a promoter sequence, said promoter sequence being operably linked to said transcription factor and being effective for the expression of a therapeutically effective amount of the transcription factor in said patient.

2. A process of claim 1, wherein said transcription factor is c-Myc.

3. A process of claim 2, wherein said DNA segment is introduced to said patient in cells, said cells having been treated in vitro to incorporate said DNA segment.

4. A process of claim 3, wherein said cells are liver cells.

5. A process of claim 3, wherein said cells are muscle cells.

6. A process of claim 3, wherein said promoter sequence is inducible during diabetic conditions.

7. A process of claim 6, wherein said promoter is a PEPCK promoter or a MLC1 promoter.

8. A process of claim 3, wherein said DNA segment has been incorporated in said cells by a viral vector.

9. A process of claim 8, wherein said viral vector is a retroviral vector.

10. A process of claim 3, wherein said cells are in a neo-organ.

11. A process of claim 10, wherein said cells are liver cells.

12. A process of claim 10, wherein said cells are muscle cells.

13. A process of claim 10, wherein said promoter sequence is inducible during diabetic conditions.

14. A process of claim 13, wherein said promoter is a PEPCK promoter or a MLC1 promoter.

15. A process of claim 2, wherein said DNA segment is administered directly to said patient.

16. A process of claim 15, wherein said DNA segment is an integral part of a vector.

17. A process of claim 16, wherein said vector is a viral vector.

18. A process of claim 17, wherein said vector is a retroviral vector, an adenoviral vector, an adeno-associated vector, a sindbis viral vector, or a herpes viral vector.

19. A process of claim 15, wherein said DNA segment is administered to said patient in a DNA ligand conjugate, a liposome, or a calcium phosphate precipitate.
